(19) United States
(12) Patent Application Publication
(10) Pub. No.: US 2022/0165365 A1
(43) Pub. Date: May 26, 2022

(54) COMPLEX ARCHITECTURE FOR REACTION CONDITION DETERMINATION

(71) Applicant: The Board of Trustees of the University of Illinois, Urbana, IL (US)

(72) Inventors: Andrew F. Zahrt, Urbana, IL (US); Nicholas Ian Rinehart, Urbana, IL (US); Scott E. Denmark, Champaign, IL (US)

(21) Appl. No.: 17/533,408

(22) Filed: Nov. 23, 2021

Related U.S. Application Data
(60) Provisional application No. 63/117,060, filed on Nov. 23, 2020.

(51) Int. Cl. G16C 20/10; G16C 20/64; G16C 20/30; G16C 20/70; G16C 20/50; G06N 3/04

(52) U.S. Cl. CPC G16C 20/10; G16C 20/64; G06N 3/04; G16C 20/70; G16C 20/50; G16C 20/30

(57) ABSTRACT

Methods, apparatus, and storage medium for determining a combination of coupling partners for a reaction according to input data. The method includes obtaining test input data for a test coupling partner of a test chemical type; obtaining selected input data for a selected coupling partner of a selected chemical type; determining, based on a reaction condition library, a candidate reaction condition set according to the test input data and selected input data, the candidate reaction condition set comprising a previous reaction condition; determining a candidate reaction vector representative of the candidate reaction condition set; inputting the candidate reaction vector into an input layer of a neural network set; and receiving an output at an output layer of the neural network set, the output indicative of a predicted yield from reacting the test coupling partner and the selected coupling partner under the candidate reaction condition set.

### RELATED APPLICATION

This invention claims the benefit of U.S. Provisional Application No. 63/117,060, filed on Nov. 23, 2020, which is incorporated by reference in its entirety.

### BACKGROUND

This disclosure relates to reaction condition determination. Rapid advances computer technologies have led to the implementation computational assistance in the operation of chemical systems. The ability of computer systems to reduce error and provide easy access to information on reaction inputs has driven demand for chemical informatics systems. Improved features in computational assistance systems will continue to drive demand.

### SUMMARY

The present disclosure relates to methods, apparatus, and non-transitory computer readable storage medium for using a neural network set to determine a reaction condition.

The present disclosure describes an apparatus for using a neural network set to determine a chemical reaction condition. The apparatus includes a memory storing instructions; and a processor in communication with the memory. When the processor executes the instructions, the processor is configured to cause the apparatus to perform: obtaining test input data for a test coupling partner of a test chemical type, obtaining selected input data for a selected coupling partner of a selected chemical type, the selected chemical type being a counterpart to the test chemical type, determining, based on a reaction condition library comprising at least one entry, a candidate reaction condition set according to the test input data and selected input data, the candidate reaction condition set comprising a previous reaction condition, determining a candidate reaction vector representative of the candidate reaction condition set, inputting the candidate reaction vector into an input layer of a neural network set, and receiving an output at an output layer of the neural network set, the output indicative of a predicted yield from reacting the test coupling partner and the selected coupling partner under the candidate reaction condition set.

The present disclosure describes a method includes a method for using a neural network set to determine a chemical reaction condition. The method may be performed by a device comprising a memory storing instructions and a processor in communication with the memory. The method includes obtaining test input data for a test coupling partner of a test chemical type; obtaining selected input data for a selected coupling partner of a selected chemical type, the selected chemical type being a counterpart to the test chemical type; determining, based on a reaction condition library comprising at least one entry, a candidate reaction condition set according to the test input data and selected input data, the candidate reaction condition set comprising a previous reaction condition; determining a candidate reaction vector representative of the candidate reaction condition set; inputting the candidate reaction vector into an input layer of a neural network set; and receiving an output at an output layer of the neural network set, the output indicative of a predicted yield from reacting the test coupling partner and the selected coupling partner under the candidate reaction condition set.

The present disclosure describes a non-transitory computer readable storage medium storing computer readable instructions. The computer readable instructions, when executed by a processor, are configured to cause the processor to perform: obtaining test input data for a test coupling partner of a test chemical type; obtaining selected input data for a selected coupling partner of a selected chemical type, the selected chemical type being a counterpart to the test chemical type; determining, based on a reaction condition library comprising at least one entry, a candidate reaction condition set according to the test input data and selected input data, the candidate reaction condition set comprising a previous reaction condition; determining a candidate reaction vector representative of the candidate reaction condition set; inputting the candidate reaction vector into an input layer of a neural network set; and receiving an output at an output layer of the neural network set, the output indicative of a predicted yield from reacting the test coupling partner and the selected coupling partner under the candidate reaction condition set.

The present disclosure also describes a system including reaction condition circuitry configured to implement any of the above methods.

The present disclosure also describes a product manufactured by any of the above methods.

### BRIEF DESCRIPTION OF THE DRAWINGS

The system, device, product, and/or method described below may be better understood with reference to the following drawings and description of non-limiting and non-exhaustive embodiments. The components in the drawings are not necessarily to scale. Emphasis instead is placed upon illustrating the principles of the present disclosure. The patent or application file contains at least one drawing executed in color. Copies of this patent or patent application publication with color drawing(s) will be provided by the Office upon request and payment of the necessary fee.

FIG. 1 shows an exemplary electronic communication environment for determining a reaction condition.

FIG. 2 shows computer systems that may be used to implement various components of the electronic communication environment of FIG. 1.

FIG. 3A shows a schematic diagram for various embodiment for determining a reaction condition.

FIG. 3A shows a schematic diagram for various embodiment for a neural network model.

FIG. 4 shows a flow diagram of an embodiment of a method for determining a reaction condition.

FIG. 5 shows a portion of a schematic diagram of a summary workflow of an exemplary embodiment.

FIG. 6 shows another portion of the schematic diagram of the summary workflow of the exemplary embodiment in FIG. 5.

FIG. 7A shows a chart of prediction of reaction outcomes for training, validation, and test sets.

FIG. 7B shows a chart of prediction of reaction outcomes for future sets.

FIG. 8A shows a summary of a reaction categorization process.

FIG. 8B shows several general reaction types in various embodiments.

FIG. 9A shows a portion of a reaction generation process.

FIG. 9B shows another portion of the reaction generation process in FIG. 9A.

FIG. 10A shows an overview of an exemplary project folder of a sample project.

FIG. 10B shows one aspect of the sample project.

FIG. 10C shows another aspect of the sample project.

FIG. 10D shows another aspect of the sample project: a sample use of a pred_from_csv_fullreactions.py script.

FIG. 11 shows an example process for submitting reaction predictions with a commandline.

FIG. 12 shows a sample submission using a csv file and output files created.

### DETAILED DESCRIPTION OF THE DISCLOSURE

The disclosed systems, devices, and methods will now be described in detail hereinafter with reference to the accompanied drawings that form a part of the present application and show, by way of illustration, examples of specific embodiments. The described systems and methods may, however, be embodied in a variety of different forms and, therefore, the claimed subject matter covered by this disclosure is intended to be construed as not being limited to any of the embodiments. This disclosure may be embodied as methods, devices, components, or systems. Accordingly, embodiments of the disclosed system and methods may, for example, take the form of hardware, software, firmware or any combination thereof.

Throughout the specification and claims, terms may have nuanced meanings suggested or implied in context beyond an explicitly stated meaning. Likewise, the phrase “in one embodiment” or “in some embodiments” as used herein does not necessarily refer to the same embodiment and the phrase “in another embodiment” or “in other embodiments” as used herein does not necessarily refer to a different embodiment. It is intended, for example, that claimed subject matter may include combinations of exemplary embodiments in whole or in part. Moreover, the phrase “in one implementation”, “in another implementation”, “in some implementations”, or “in some other implementations” as used herein does not necessarily refer to the same implementation(s) or different implementation(s). It is intended, for example, that claimed subject matter may include combinations of the disclosed features from the implementations in whole or in part.

In general, terminology may be understood at least in part from usage in context. For example, terms, such as “and”, “or”, or “and/or,” as used herein may include a variety of meanings that may depend at least in part upon the context in which such terms are used. In addition, the term “one or more” or “at least one” as used herein, depending at least in part upon context, may be used to describe any feature, structure, or characteristic in a singular sense or may be used to describe combinations of features, structures or characteristics in a plural sense. Similarly, terms, such as “a”, “an”, or “the”, again, may be understood to convey a singular usage or to convey a plural usage, depending at least in part upon context. In addition, the term “based on” or “determined by” may be understood as not necessarily intended to convey an exclusive set of factors and may, instead, allow for existence of additional factors not necessarily expressly described, again, depending at least in part on context.

The present disclosure relates to methods for using a neural network set to determine a chemical reaction condition. The neural network set may include a single neural network, or may include a plurality of neural networks as an ensemble of neural networks.

In various contexts, catalogued (and/or catalogable) knowledge of potential chemical reaction conditions for various types of reactants may be available to guide selection of reaction conditions. In some cases, systematic treatment of such reaction data may help to guide selection possible reaction conditions where a combination of reactants may be novel (e.g., previously unreported) but data is available of reactions successfully executed with reactants of the same type as the novel combination.

In some cases, machine-learning models may be developed trained to reproduce (at least in part) reaction condition data relating reaction condition inputs (e.g., reactants and/or other reaction components) to reaction outputs (e.g., yields or other reaction outputs). The models then may be used to predict outputs for input combinations that may not necessarily be included within the previously cataloged reactions.

More detailed description of reaction library, chemical structures, neural networks and other related topics is included in U.S. application Ser. No. 16/551,007 filed on Aug. 26, 2019 by the same Applicant as the present application, which is incorporated herein by reference in its entirety.

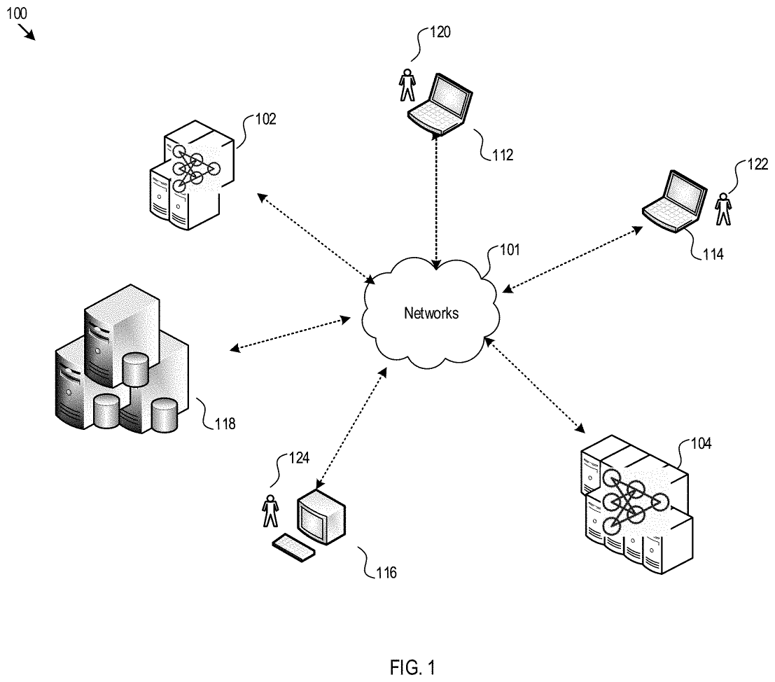

FIG. 1 shows an exemplary electronic communication environment 100 in which one or more embodiments of determining at least one reaction condition may be implemented. The electronic communication environment 100 may include a portion or all of the following: one or more servers (102 and 104) implementing the embodiment of a neural network set, one or more user devices (112, 114, and 116) associated with users (120, 122, and 124), and one or more databases 118, in communication with each other via public or private communication networks 101.

The server 102 may be implemented as a central server or a plurality of servers distributed in the communication networks. While the server 102 shown in FIG. 1 is implemented as a single server, the server 102 may be implemented as a group of distributed servers, or may be distributed on the server 104.

The user devices 112, 114, and 116 may be any form of mobile or fixed electronic devices including but not limited to desktop personal computer, laptop computers, tablets, mobile phones, personal digital assistants, and the like. The user devices 112, 114, and 116 may be installed with a user interface for accessing the embodiment for determining at least one reaction condition. The one or more database 118 of FIG. 1 may be hosted in a central database server, a plurality of distributed database servers, or in cloud-based database hosts. The database 118 may be organized and implemented in any form, including but not limited to relational database containing data tables, graphic database containing nodes and relationships, and the like. The database 118 may be configured to store the intermediate data and/or final results for implementing the embodiment for determining at least one reaction condition.

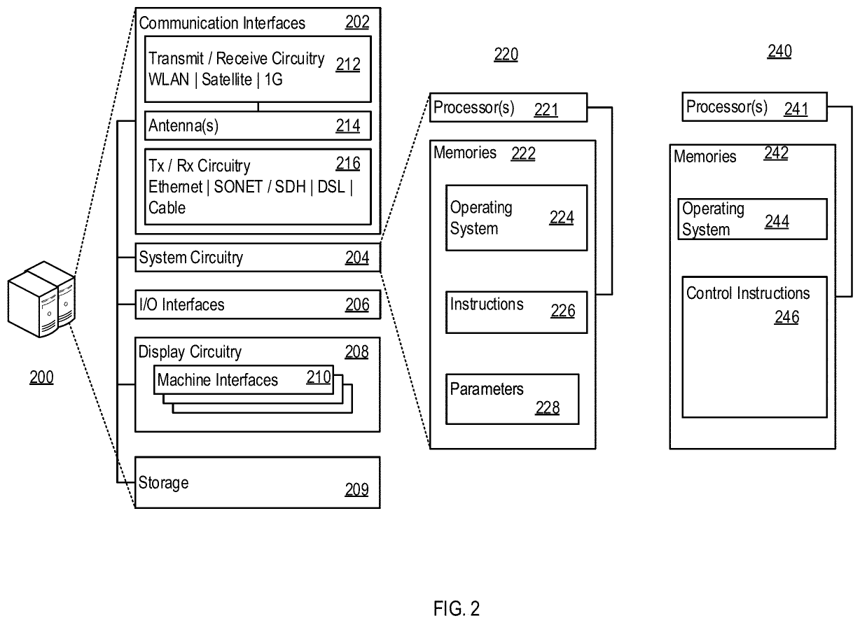

FIG. 2 shows an exemplary system, which is a computer system 200 for implementing the server 102, or the user devices 112, 114, and 116. The computer system 200 may include communication interfaces 202, system circuitry 204, input/output (I/O) interfaces 206, storage 209, and display circuitry 208 that generates machine interfaces 210 locally or for remote display, e.g., in a web browser running on a local or remote machine. The machine interfaces 210 and the I/O interfaces 206 may include GUIs, touch sensitive displays, voice or facial recognition inputs, buttons, switches, speakers and other user interface elements. Additional examples of the I/O interfaces 206 include microphones, video and still image cameras, headset and microphone input/output jacks, Universal Serial Bus (USB) connectors, memory card slots, and other types of inputs. The I/O interfaces 206 may further include magnetic or optical media interfaces (e.g., a CDROM or DVD drive), serial and parallel bus interfaces, and keyboard and mouse interfaces.

The communication interfaces 202 may include wireless transmitters and receivers (“transceivers”) 212 and any antennas 214 used by the transmitting and receiving circuitry of the transceivers 212. The transceivers 212 and antennas 214 may support Wi-Fi network communications, for instance, under any version of IEEE 802.11, e.g., 802.11n or 802.11ac. The communication interfaces 202 may also include wireline transceivers 216. The wireline transceivers 116 may provide physical layer interfaces for any of a wide range of communication protocols, such as any type of Ethernet, data over cable service interface specification (DOCSIS), digital subscriber line (DSL), Synchronous Optical Network (SONET), or other protocol.

The storage 209 may be used to store various initial, intermediate, or final data or model for implementing the embodiment for determining at least one reaction condition. These data corpus may alternatively be stored in the database 118 of FIG. 1. In one implementation, the storage 209 of the computer system 200 may be integral with the database 118 of FIG. 1. The storage 209 may be centralized or distributed, and may be local or remote to the computer system 200. For example, the storage 209 may be hosted remotely by a cloud computing service provider.

The system circuitry 204 may include hardware, software, firmware, or other circuitry in any combination. The system circuitry 204 may be implemented, for example, with one or more systems on a chip (SoC), application specific integrated circuits (ASIC), microprocessors, discrete analog and digital circuits, and other circuitry.

For example, at least some of the system circuitry 204 may be implemented as processing circuitry 220 for the server 102 in FIG. 1. The processing circuitry 220 may include one or more processors 221 and memories 222. The memories 222 stores, for example, control instructions 226, parameters 228, and/or an operating system 224. The control instructions 226, for example may include instructions for implementing various components of the embodiment for determining at least one reaction condition. In one implementation, the instruction processors 221 execute the control instructions 226 and the operating system 224 to carry out any desired functionality related to the embodiment for determining at least one reaction condition.

Alternatively, or in addition, at least some of the system circuitry 204 may be implemented as client circuitry 240 for the user devices 112, 114, and 116 of FIG. 1. The client circuitry 240 of the user devices may include one or more instruction processors 241 and memories 242. The memories 242 stores, for example, control instructions 246 and/or an operating system 244. In one implementation, the instruction processors 241 execute the control instructions 246 and the operating system 244 to carry out any desired functionality related to the user devices.

In some embodiments, a method for determining at least one chemical reaction condition may be implemented in a device comprising memory storing instructions and a processor in communication with the memory, for example, a computer. The memory may be used to store one or more parameters, hyperparameters, and/or model templates used in machine learning models. The memory may further store validity rules, that may facilitate selection of candidate reaction conditions within the validity domain. The memory may further include applications and structures, for example, coded objects, templates, or one or more other data structures to the reaction condition analysis. The processor may execute the instruction in the memory to implement reaction condition logic, which may provide software support to implement the various tasks performed by the system. The device may further include a user interface that may include man-machine interfaces and/or graphical user interfaces (GUI). The GUI may be used to present interfaces and/or options to operators involved in input of reactant data and/or viewing model results.

In various implementations, the trained machine-learning models may have an associated validity domain. For example, the machine-learning models may provide prediction for combinations of reactants that are similar to those captured in the reaction condition data used to train the machine learning model. In an illustrative example discussed below the reaction data includes electrophile-nucleophile couplings. Accordingly, the model trained used that data may be focused on other such couplings. In various embodiments, a nucleophile may be also referred as a silicon-containing starting material. Further, the electrophiles and nucleophile chemical types are further divided into type-classes (e.g., bromine containing electrophiles, iodine containing electrophiles, chlorine containing electrophiles, sulfonate containing electrophiles for the electrophiles; and aryl nucleophiles, heteroaryl nucleophiles, alkenyl nucleophiles, and alkynyl nucleophiles for the nucleophiles). Accordingly, in the illustrative example, the validity domain for reaction conditions for which a prediction may be made by the machine learning model may include domains for which the reactants match in type and/or type-class.

Various machine learning model types may be used to predict outputs for various different conditions for (e.g., user selected, or otherwise selected) reactants. As an example, a multiple layer neutral network (e.g., with input, output, and one or more hidden layers) may serve as the machine-learning model. The inputs (e.g., the candidate reaction condition set) may be coded as reaction vectors (e.g., using bit vectors) to represent the presence or absence of various reaction components. In the illustrative example with nucleophiles and electrophiles, example reaction components may include reactants, equivalencies, Pd-sources, ligands, bases, solvents, additives, reaction temperatures, products, yields and/or other reaction conditions. The reaction vectors may be applied to the input layer nodes of the neural network. In various implementations, the dimensionality of the vector may match the number of nodes present in the input layer. Various other input structures may be used.

In various implementations, the predicted yield may be displayed on interactive interface display elements to facilitate user control. In some cases, multiple candidate reaction conditions may be ranked relative to one another for a given set of reactant inputs (e.g., provided by the user via the interactive interface display).

The methods, devices, processing, and logic described above may be implemented in many different ways and in many different combinations of hardware and software. For example, all or parts of the implementations may be circuitry that includes an instruction processor, such as a Central Processing Unit (CPU), microcontroller, or a microprocessor; an Application Specific Integrated Circuit (ASIC), Programmable Logic Device (PLD), or Field Programmable Gate Array (FPGA); or circuitry that includes discrete logic or other circuit components, including analog circuit components, digital circuit components or both; or any combination thereof. The circuitry may include discrete interconnected hardware components and/or may be combined on a single integrated circuit die, distributed among multiple integrated circuit dies, or implemented in a Multiple Chip Module (MCM) of multiple integrated circuit dies in a common package, as examples.

The circuitry may further include or access instructions for execution by the circuitry. The instructions may be embodied as a signal and/or data stream and/or may be stored in a tangible storage medium that is other than a transitory signal, such as a flash memory, a Random Access Memory (RAM), a Read Only Memory (ROM), an Erasable Programmable Read Only Memory (EPROM); or on a magnetic or optical disc, such as a Compact Disc Read Only Memory (CDROM), Hard Disk Drive (HDD), or other magnetic or optical disk; or in or on another machine-readable medium. A product, such as a computer program product, may particularly include a storage medium and instructions stored in or on the medium, and the instructions when executed by the circuitry in a device may cause the device to implement any of the processing described above or illustrated in the drawings.

The implementations may be distributed as circuitry, e.g., hardware, and/or a combination of hardware and software among multiple system components, such as among multiple processors and memories, optionally including multiple distributed processing systems. Parameters, databases, and other data structures may be separately stored and managed, may be incorporated into a single memory or database, may be logically and physically organized in many different ways, and may be implemented in many different ways, including as data structures such as linked lists, hash tables, arrays, records, objects, or implicit storage mechanisms. Programs may be parts (e.g., subroutines) of a single program, separate programs, distributed across several memories and processors, or implemented in many different ways, such as in a library, such as a shared library (e.g., a Dynamic Link Library (DLL)). The DLL, for example, may store instructions that perform any of the processing described above or illustrated in the drawings, when executed by the circuitry.

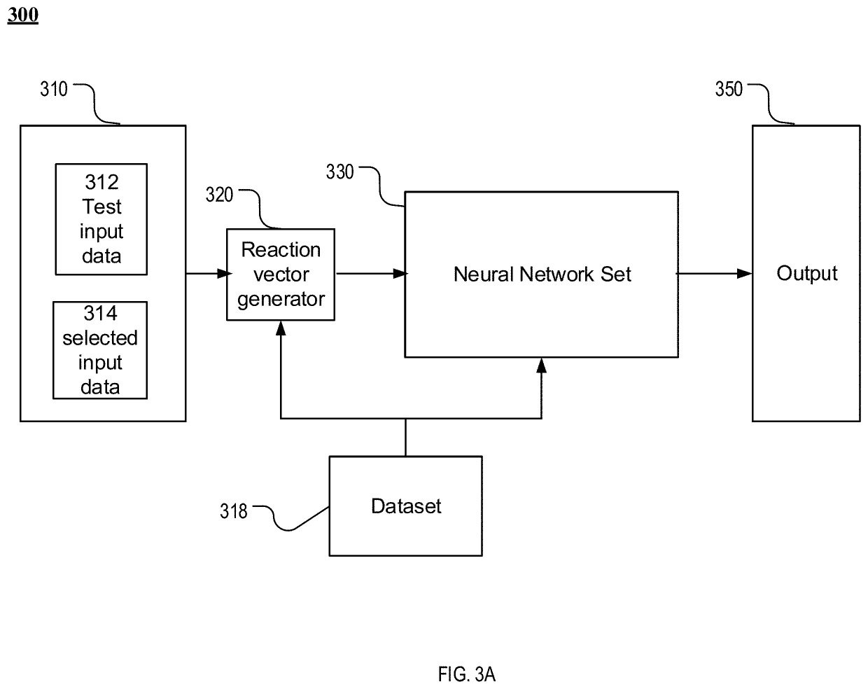

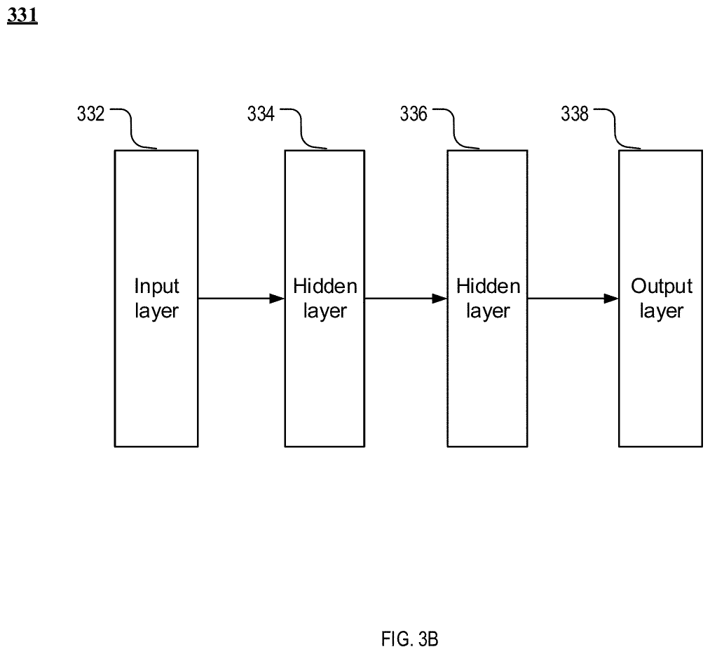

FIG. 3A shows a schematic diagram of an embodiment of a system for using a neural network set to determine a reaction condition. The system 300 may include a portion or all of the following: a neural network set 330, an input 310, an output 350, a dataset 318, and/or a reaction vector generator 320. The dataset 318 may include a reaction condition library.

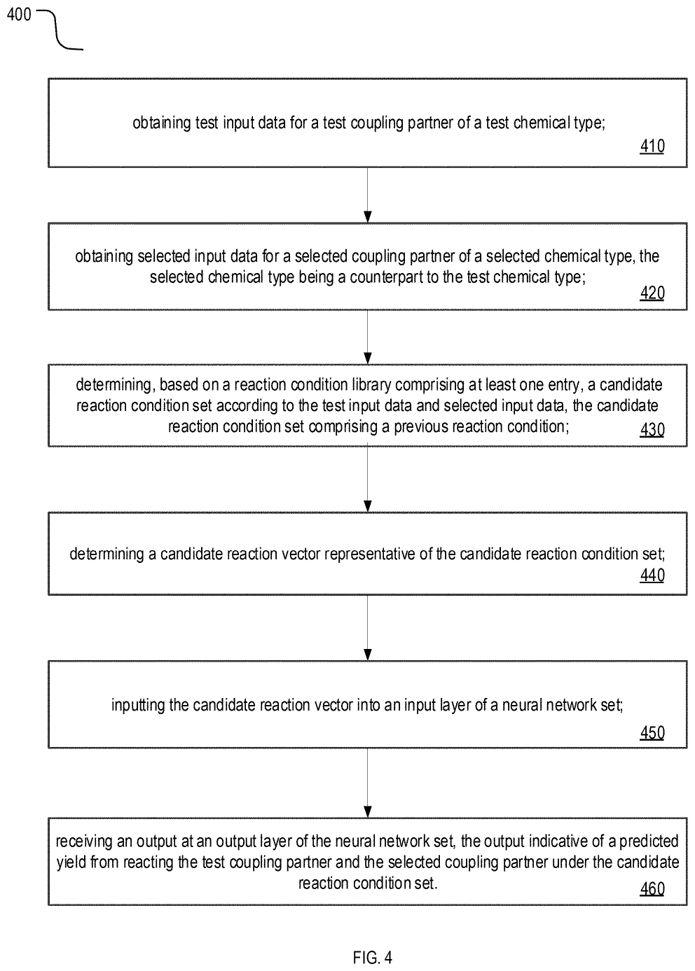

Referring to FIG. 4, the present disclosure describes various embodiments of a method 400 of using a neural network set to determine a reaction condition. The method 400 may include a portion or all of the following steps: step 410, obtaining test input data for a test coupling partner of a test chemical type; step 420, obtaining selected input data for a selected coupling partner of a selected chemical type, the selected chemical type being a counterpart to the test chemical type; step 430, determining, based on a reaction condition library comprising at least one entry, a candidate reaction condition set according to the test input data and selected input data, the candidate reaction condition set comprising a previous reaction condition; step 440, determining a candidate reaction vector representative of the candidate reaction condition set; step 450, inputting the candidate reaction vector into an input layer of a neural network set; and/or step 460, receiving an output at an output layer of the neural network set, the output indicative of a predicted yield from reacting the test coupling partner and the selected coupling partner under the candidate reaction condition set.

Referring back to FIG. 3A, the input 310 may include test input data 312 and selected input data 314. The test input data 312 may be for a test coupling partner of a test chemical type, and/or the selected input data 314 may be for a selected coupling partner of a selected chemical type. For example, the test chemical type comprises a nucleophile chemical type, and/or the selected chemical type includes an electrophile chemical type. The selected chemical type may be a counterpart to the test chemical type, for example, the test coupling partner and the selected coupling partner may couple via a sp2-sp2 coupling and/or a sp-sp2 coupling.

In some implementations, the test/selected input data may include (or be represented in a form of) a simplified molecular-input line-entry system (SMILES) string or other line-entry string for a chemical model. In some other implementations, when the test/selected input data include two-dimensional or three-dimensional model of a chemical model, the method 400 may include converting the multi-dimensional model to a line-entry string for the chemical model.

The dataset 318 may include a reaction condition library comprising one or more entry. For example, the dataset may include a portion of or all entries from an Organic Reactions (OR) chapter, and/or each entry of the dataset includes at least one of the following: reactants, equivalencies, Pd-sources, ligands, base, solvent(s), additives, reaction temperature, products, and/or yield.

The reaction vector generator 320 may determine, based on the dataset including a reaction condition library, a candidate reaction condition set according to the test input data and selected input data, the candidate reaction condition set comprising a previous reaction condition; and determine a candidate reaction vector representative of the candidate reaction condition set. In some implementations, the previous reaction condition may include at least one of the following: an equivalency, a Pd-source, a ligand, a base, a solvent, an additive, a reaction temperature, a product, and/or a yield.

In some implementations, the candidate reaction vector may have more than one dimensions and may be represented as a bit vector representing a presence or an absence of one reaction component. In some other implementations, the candidate reaction vector may have hundreds of dimensions, for example, having a dimension of 358.

In some other implementation, the reaction vector generator 320 may determine the candidate reaction vector and assign the candidate reaction condition set to one or more bit vectors indicating the presence or absence of reaction conditions. In one implementation, the reaction vector generator 320 may determine the candidate reaction vector by concatenating the bit vectors or adding the bit vectors to obtain an information-preserving data-structure. In another implementation, the reaction vector generator 320 may assign the bit vectors by using virtually any encoding scheme that may represent the possible reaction conditions for the bit vectors, calculating 128-bit Morgan fingerprints for reactants, omitting descriptor for products, assigning a bit vector with a specific dimension, and/or using a one-hot/cold encoding scheme by using a single first-state bit with other encoding bits in the other state.

The neural network set 330 may include a single neural network model or an ensemble of more than one neural network models. For example, the neural network set may include an ensemble of 100 different neural networks. An averaged output of the ensemble of neural networks may be used as a predicted value, as a portion of the output 350. The standard deviation in predicted values from the ensemble of neural networks may be used to reflect a confidence in the predication, as another portion of the output 350.

FIG. 3B shows an exemplary implementation of the single neural network model or one neural network model (331) of the ensembles of more than one neural network models. The neural network 331 may include a portion or all of the following: an input layer 332, one or more hidden layer (e.g., an implementation including two hidden layers 334 and 336), and/or an output layer 338. For example, the input layer may include 358 neurons, which matches to a candidate reaction vector having a dimension of 358. For another example, the output layer may include a single neuron.

For the ensemble of neural networks, hyperparameters may be optimized for each neural network in the ensemble of neural networks. The hyperparameters may include at least one of the following: activation functions, percent dropout in the first and/or second hidden layer, and/or a number of nodes in the one or more hidden layer. For example, activation functions may include at least one of the following: Relu, Selu, and/or Elu functions. A number of nodes for the one or more hidden layer may be in a range between 2 and 357, inclusive. A dropout for each layer may be within a range between 0 and 0.3, inclusive.

The neural network set 330 may receive the candidate reaction vector as input into an input layer of a neural network set; and may output at an output layer of the neural network set, the output indicative of a predicted yield from reacting the test coupling partner and the selected coupling partner under the candidate reaction condition set.

In some embodiments, the neural network set 330 may further rank the candidate reaction condition set based on the predicted yield relative to other possible reaction condition sets for the test coupling partner and selected coupling partner. In some implementations, the other possible reaction condition sets are reaction conditions sets within the validity domain.

In some embodiments, the method 400 may optionally and/or alternatively include one or more of the following: determining, based the reaction condition library, a type-class for the test coupling partner, the selected coupling partner, or both; determining a validity domain based on the type-class; determining a candidate reaction condition set, the candidate reaction condition set optionally including a previous reaction condition used in a previous reaction involving a member of the test chemical type and a member of the selected chemical type; and/or rejecting a second reaction condition set responsive to the second reaction condition set being outside the validity domain; comparing the candidate reaction condition set to previous reaction condition sets by applying the candidate condition vector to an input layer of a neural network.

In some embodiments, the method 400 may optionally and/or alternatively include one or more of the following: determining a test type-class for the test coupling partner, the test type-class comprising a type-class within the test chemical type; and/or determining a selected type-class for the selected coupling partner, the selected type-class comprising a type-class within the selected chemical type. In some implementations, determining the selected type-class, the test type-class, or both comprises referencing the reaction condition library; and/or determining the selected type-class, the test type-class, or both comprises analyzing a chemical structure specified in the test input data, the selected input data, or both.

In some other implementations, when the test coupling partner is a member of two or more type-classes, a highest ranked type-class may be selected as the test type-class for the test coupling partner. For example, the test type-class may include at least one of the following: an aryl nucleophile, a heteroaryl nucleophile, an alkenyl nucleophile, or alkynyl nucleophile.

In some embodiments, the method 400 may optionally and/or alternatively include subdividing the test type-class into test type-subclasses; determining test type-subclass for the test coupling partner includes determining a silicon-containing moiety for the test coupling partner; assigning the test coupling partner to a test type-class based on the silicon-containing moiety. For example, the silicon-containing moiety includes at least one of the following: a trimethylsilyl silicon-containing moiety, a dimethyl silanol silicon-containing moiety, or a triallylsilyl silicon-containing moiety, or other silicon-containing moiety.

In some other implementations, when the selected coupling partner is a member of two or more type-classes, a highest ranked type-class of which that the selected coupling partner is a member may be selected as the selected type-class for the selected coupling partner.

In some other implementations, the selected type-class may include at least one of the following: bromine containing electrophiles, iodine containing electrophiles, chlorine containing electrophiles, sulfonate containing electrophiles, or any combination thereof. In some other implementations, the iodine containing electrophiles are ranked higher than the bromine containing electrophiles; the bromine containing electrophiles are ranked higher than the chlorine containing electrophiles; and/or the chlorine containing electrophiles are ranked higher than the sulfonate containing electrophiles.

Referring back to FIG. 3A, the neural network set 330 may be trained using reaction condition sets for previous chemical reactions in the reaction condition library. For one example, a dataset may include about 1450 reaction conditions, which may be divided into 1110 training reactions, 185 validation reactions, and 155 test reactions. The training reactions may be used to train the neural network set by performing a number of iterations, for example, 1000 iterations, or to reach a certain threshold. The validation reactions may be used to validate the neural network set; and/or the test reactions may be used to test the neural network set. An exemplary implementation of training/validating/testing the neural network set is described in details in the later sections of the present disclosure.

### Exemplary Embodiment for Determining Reaction Condition

An example of a machine learning (“ML”) tool of the present disclosure may directly benefit bench chemists working in the laboratory. If literature data is to be used to successfully generate useful machine learning methods, (1) a prediction should be limited such that the prediction falls within a domain of a model; (2) a rapidly calculable molecular representation should be employed to allow for rapid predictions in new systems; and/or (3) a user with no programming experience should be able to use a system to efficiently make predictions. To develop the examples of machine learning tools of the present disclosure, palladium-catalyzed, silicon-based, cross-coupling reactions were identified as a model system library. Palladium-catalyzed, silicon-based, cross-coupling reactions represent an attractive prototype library application due to considerable experience and expertise of the present researchers regarding the efficacies of various silicon-group-substituted substrates and cross-coupling reaction conditions, as well as the availability of comprehensive literature on palladium-catalyzed, silicon-based, cross-coupling reactions for tabulation and use for machine learning applications. Using the available literature data from a chapter of Organic Reactions (OR), machine learning models were developed that could accurately predict cross-coupling reaction yield. The machine learning models capable of accurately predicting cross-coupling reaction yield were incorporated into a recommender system designed to identify effective reaction conditions for novel palladium-catalyzed, silicon-based, cross-coupling reactions. Reaction categorization and expert knowledge is used to constrain permissible predictions of the recommender system to conditions similar to the conditions used in construction of the machine learning models. Accordingly, the recommender system avoids extrapolating into unrepresented chemical space, thereby increasing prediction accuracy of recommended reaction conditions.

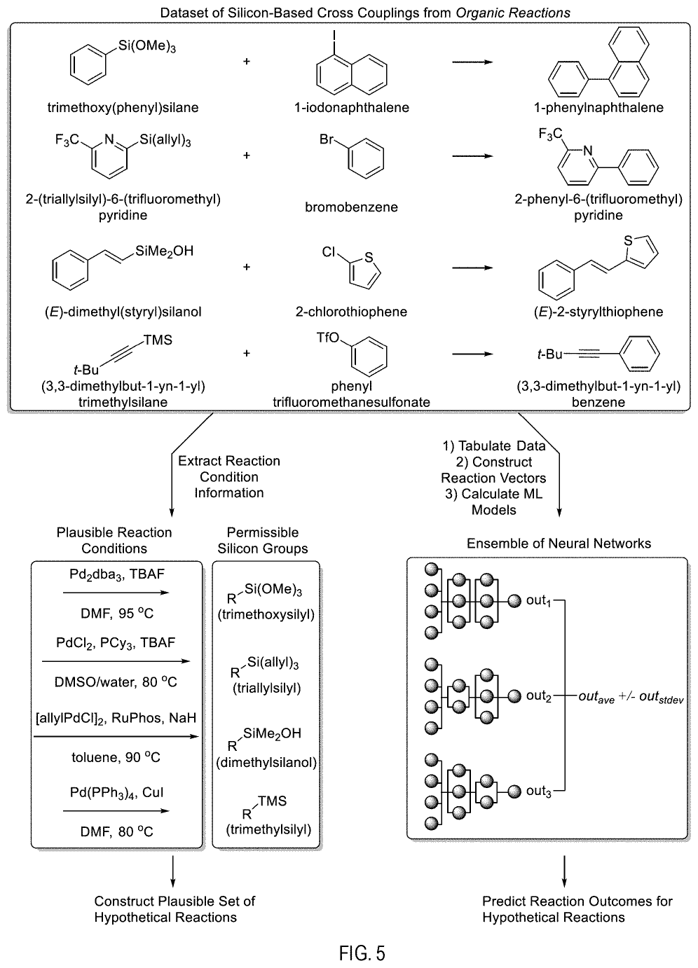

As illustrated by exemplary data in FIG. 5, initially, literature data was manually curated, by encoding silanes (as shown in FIG. 5, trimethoxy(phenyl)silane, 2-(triallylsilyl)-6-(trifluoromethyl)pyridine, (E)-dimethyl(styryl)silanol, and (3,3-dimethylbut-1-yn-1-yl)trimethylsilane); electrophiles (as shown in FIG. 5, 1-iodonaphthalene, bromobenzene, 2-chlorothiophene, and phenyl trifluoromethanesulfonate); stoichiometric equivalencies; palladium sources (as shown in FIG. 5, tris(dibenzylideneacetone)dipalladium(0) (Pd₂(dba)₃), palladium (II) chloride, allylpalladium (II) chloride dimer (allylPdCl)₂), and tetrakis(triphenylphosphine)palladium(0) (Pd(PPh₃)₄)); catalyst ligands (as shown in FIG. 5, tricyclohexylphosphine and RuPhos); alkaline co-reagents (as shown in FIG. 5, sodium hydride (NaH)); solvent(s) (as shown in FIG. 5, dimethylformamide (DMF), dimethylsulfoxide (DMSO), water, and toluene); additive(s) (as shown in FIG. 5, tetrabutylammonium fluoride (TBAF) and cuprous iodide (CuI); reaction temperatures (as shown in FIG. 5, 80° C., 90° C., and 95° C.); cross-coupling products (as shown in FIG. 5, 1-phenylnaphthalene, 2-phenyl-6-(trifluoromethyl)pyridine, (E)-2-styrylthiophene, and (3,3-dimethylbut-1-yn-yl)benzene); permissible silicon groups (as shown in FIG. 5, trimethoxysilyl, triallylsilyl, dimethylsilanol, and trimethylsilyl); and yield. Only cross-couplings between sp- or sp²-hybridized silanes (i.e., carbon bonded to silicon is sp- or sp²-hybridized) and sp- or sp²-hybridized electrophiles (i.e., carbon bonded to leaving group is sp- or sp²-hybridized) were included in the curation, so as to construct a dataset of approximately 1400 cross-coupling reactions. Reaction vectors were then constructed by linking together representations for different reaction components, after extracting reaction conditions information, including plausible reaction conditions for each possible silyl group. For the representations of palladium sources, catalyst ligands, alkaline co-reagents, solvent(s), and additive(s), the different components were represented as bit vectors denoting a presence or an absence of each reaction component (one-hot encoding). Pairs of coupling starting materials (a silane and an electrophile) may be used with a molecular representation that enables the prediction of reaction outcomes with novel pairs of coupling starting materials by using Morgan Fingerprints to represent the silane coupling starting material and the electrophile starting material.

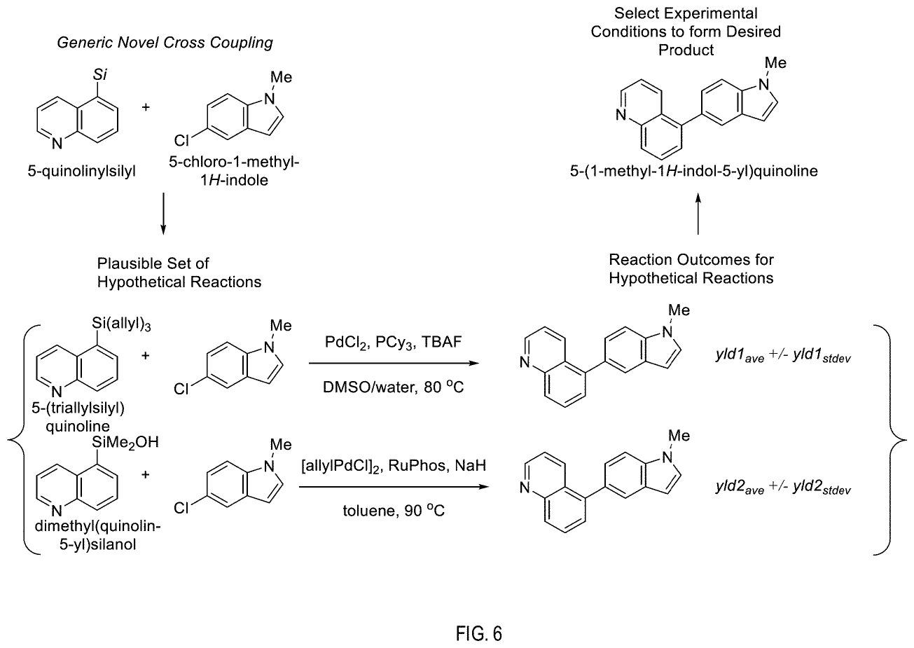

The example of the system input illustrated in FIG. 5 may provide predictions of reaction outcomes and reaction conditions for different silane and electrophile pairs of coupling starting materials, with a metric of confidence in the predictions. For example, as illustrated in FIG. 6, a user of a recommender system may wish to determine the reaction outcome and reaction conditions for a generic, novel palladium-catalyzed cross-coupling reaction between a 5-quinolinyl silyl group compound and 5-chloro-1-methyl-1H-indole. From the plausible reaction conditions for each possible silicon group as illustrated by example in FIG. 5, a plausible set of hypothetical reactions may be constructed. For example, as shown in FIG. 6, the 5-quinolinyl silyl group compound may be 5-(triallylsilyl)quinoline, and based on the triallylsilyl group, the hypothetical reaction of 5-(triallylsilyl)quinoline and 5-chloro-1-methyl-1H-indole would include palladium (II) chloride (PdCl₂), tricyclohexylphosphine (PCy₃), tetrabutylammonium fluoride (TBAF), dimethylsulfoxide (DMSO) and water, at 80° C. The hypothetical reaction would provide a predicted reaction outcome of producing 5-(1-methyl-1H-indol-5-yl)quinoline at a particular yield. Alternatively, the 5-quinolinyl silyl group compound may be dimethyl(quinolin-5-yl)silanol, and based on the dimethylsilanol group, the hypothetical reaction of dimethyl(quinolin-5-yl)silanol and 5-chloro-1-methyl-1H-indole would include allylpalladium (II) chloride dimer (allylPdCl)₂), RuPhos, sodium hydride (NaH), and toluene, at 90° C. The hypothetical reaction would provide a predicted reaction outcome of producing 5-(1-methyl-1H-indol-5-yl)quinoline at a particular yield.

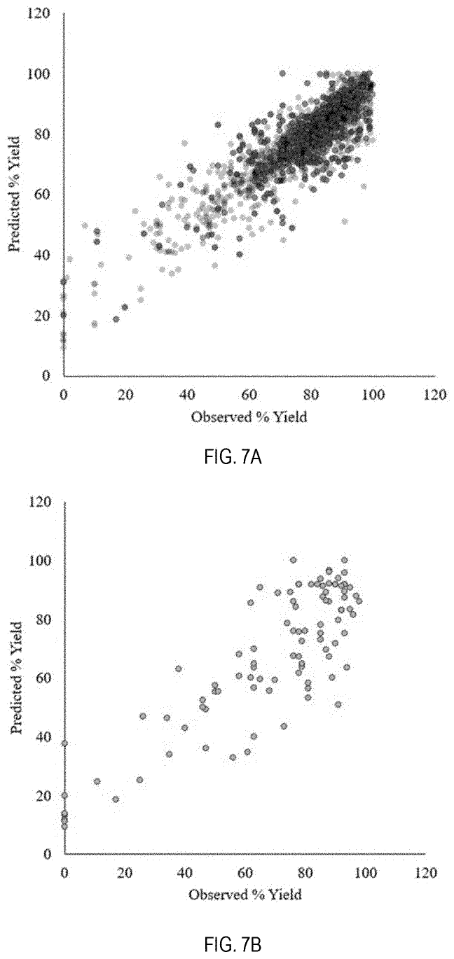

Ensembling methods, using numerous estimators, may provide some indication of prediction accuracy. In the study illustrated in FIGS. 5 and 6, an ensemble of 100 different neural networks was constructed, with an average output of those networks used as a predicted value. The standard deviation in the predicted value may reflect the confidence in a prediction; if all estimators are in agreement regarding an outcome, the outcome likely has greater certainty than a reaction in which there is substantial disagreement among estimators. By dividing the approximately 1400 reaction dataset into training (including 1110 reactions), validation (including 185 reactions), and test (including 155 reactions) subsets, models were constructed with excellent accuracy in predicting yields of reactions (Mean Absolute Error (MAE)_{Train}=5.7%, MAE_{Validation}=8.4%, MAE_{Test}=7.8%, illustrated in FIG. 7A). As a more rigorous evaluation of the models, a set of 97 reactions published subsequent to 2012 were collected to simulate the target use of the recommender system. Because the Organic Reactions chapter was published in 2011, the 97 reactions published after 2012 constitute “future” predictions with respect the models; in other words, the 97 reactions published after 2012 had not been run at the time the data used to generate the models was collected experimentally. The same ensemble of networks that demonstrated the accuracy of yield prediction in FIG. 7A was used to accurately predict the yield of the 97 reactions published, with MAE_{Future}=11.0%—(illustrated in FIG. 7B), thereby demonstrated the potential to use the examples of models of the present disclosure to predict optional reaction conditions in future applications.

As illustrated in FIGS. 7A and 7B, the example of an ensemble of neural networks of the present disclosure demonstrates excellent predictive power, accurately predicting the yield of palladium-catalyzed, silicon-based cross-coupling reactions. FIG. 7A illustrates prediction of reaction outcomes for the training set of 1110 reactions (grey/black datapoints), the validation set of 185 reactions (blue datapoints), and the external test set of 155 reactions (red datapoints). FIG. 7B illustrates the set of “future” (post-2012) 97 reactions (yellow datapoints). In the data, there are only a few examples of negative reaction results. Accordingly, the dataset is skewed such that most data points indicate high yield. Thus, though the model generally predicts the correct rank-order of reactions, which is the most critical aspect of an effective recommender tool), the model systematically “over predicts” or overestimates the reaction outcomes of low-yielding reactions. While the model avoids predicting a reaction with less than 20% measured yield to yield greater than 85%, reactions observed experimentally to yield 0% cross-coupling product may be predicted to have as high as approximately 40% yield. Further, unsuccessful reaction conditions are typically not reported in literature, so the system cannot identify unreasonable reaction conditions that are not included in the dataset. For example, a hypothetical cross-coupling reaction performed without a catalyst ligand at room temperature on an aryl chloride electrophile may be predicted by the model to have a non-zero yield; an expert would identify the hypothetical cross-coupling reaction as an unwise selection of reaction conditions. It was hypothesized that a reaction prediction system could be devised that avoids the pitfalls of overestimation and inability to identify unreasonable reaction conditions. Accordingly, the present disclosure provides a system that uses the synergy between expert knowledge of selecting reaction conditions and machine learning to recommend reaction conditions for novel cross coupling reactions.

In accordance with an example of a machine learning model, the present disclosure provides a system using reaction classification and expert knowledge to constrain a domain of future predictions to an applicability domain of the model. To use the system, a user provides a generic silicon-containing starting material, wherein only the silicon-containing moiety is undefined, and an electrophile, as in the example shown in FIG. 6 of the 5-quinolinyl silyl group compound and 5-chloro-1-methyl-1H-indole, respectively. The system then generates a plurality of silicon-containing starting materials of the same structure as the generic silicon-containing starting material, the silicon-containing starting materials differing only in the identity of the silicon-containing moiety, similarly to how the plurality of silicon-containing starting materials in FIG. 6 include 5-(triallylsilyl)quinoline and dimethyl(quinolin-5-yl)silanol having the same structure as the generic silicon-containing starting material 5-quinolinylsilyl shown in FIG. 6. The system also generates a series of reasonable reaction conditions for the coupling of each particular silicon-containing moiety with the electrophile. The system then predicts reaction outcomes for all of the in silico reactions, outputting a yield predicted by the ensemble of neural networks, and a standard deviation in the yield as an accuracy metric. The system may identify a plurality of reasonable reaction conditions for a particular novel coupling while mitigating extension into unrepresented regions of reaction space. In various embodiments, a silicon-containing starting material may be also referred as a nucleophile.

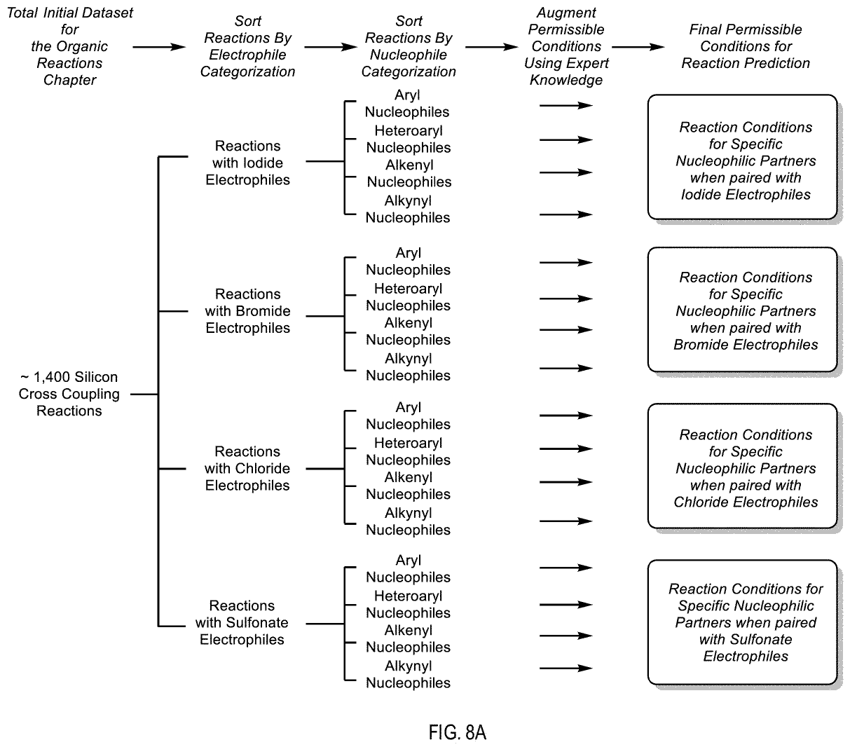

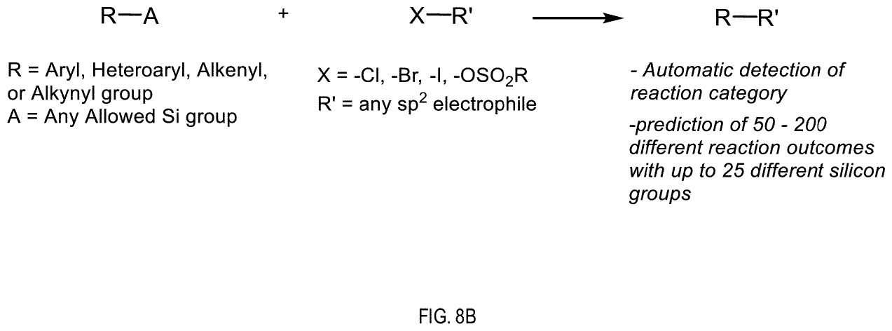

To implement this system, the system may first identify the silicon-containing starting material and the electrophile and then suggests plausible conditions for the cross-coupling reaction. To enable the system to identify the starting materials and suggest conditions, the reactions in the approximately 1400-reaction database were divided first by the identity of the leaving group of the electrophile into four different groups: iodides, bromides, chlorides, and sulfonates. The reactions in each of the four categories of leaving groups of the electrophiles were then subdivided into four subcategories silicon-containing starting material based on the moiety including the sp- or sp²-hybridized carbon bonded to silicon: aryl, heteroaryl, alkenyl, and alkynyl. The reactions in each of the subcategories were further subdivided into subsubcategories on the basis of the particular silicon-containing moiety in the silicon-containing starting material, e.g.: trimethylsilyl, dimethylsilanol, and triallylsiyl. A summary of the reaction categorization process used to construct the pool of permissible reactions for particular reaction types is illustrated in FIG. 8A, including the division and subdivision of the reaction database.

Some subcategories or subsubcategories did not include any reactions. For example, if a certain silicon-containing moiety was in a particular silicon-containing starting material that was included in the reaction database only in couplings with aryl iodides, no reaction conditions would be provided as predictions in reactions of the particular silicon-containing starting material with aryl chloride electrophiles. In such cases, the silicon-containing starting material including the silicon-containing moiety would be omitted as a supported option, or conditions would be manually or automatically added from a different silicon-containing moiety requiring a similar type of activation. For example, if a silicon-containing moiety requiring fluoride activation is not represented by any database reactions, reaction conditions may be borrowed from a different silicon-containing moiety requiring fluoride activation if the different silicon-containing moiety has been used with the same type of silicon-containing starting material (aryl, heteroaryl, alkenyl, or alkynyl) and electrophile.

The above protocol yields a final set of reaction conditions that may be predicted by the model, an example of which is illustrated in FIG. 8A. Future predictions may employ reaction conditions that have been used previously for general classes of silicon-containing starting materials and electrophiles, while the domain of future predictions may exclude untenable reaction predictions that are absent from the training data. By this method, the system mitigates the gaps in the literature data set of reactions, enabling the construction of a useful machine learning tool without experimental overhead.

Once the set of allowable reaction conditions for each type of silicon-containing starting material and electrophile are identified, the program will use these possibilities to construct a set of virtual reactions for each desired cross-coupling reaction, and predict the experimental outcome. The system is designed to take as input a desired silicon-containing starting material with a dummy atom (denoted “A”) as a placeholder for any permissible silicon-containing moiety a particular category of silicon-containing starting material as a SMILES string, as illustrated in FIG. 8B, which provides the general starting material types supported by the predictor model of the present disclosure. The system also requires input of the electrophile as a SMILES string. Both inputs can be simply drawn in Chem Draw chemical drawing software, copied as a SMILES string, and pasted into the input directly. The system may then generate up to 25 unique nucleophiles with different silicon-containing moiety and predict between approximately 50 and 200 different reaction conditions, each generating the same cross-coupling product, in a few seconds of time. The outputs may be exported to a csv file, which may be rank-ordered by predicted yield or prediction confidence, as desired.

### Dataset Construction

In an exemplary embodiment, a dataset used in the present disclosure may be manually tabulated from the Organic Reactions chapter. In the data tabulation process, only sp-sp² and sp²-sp² couplings may be recorded. Further, certain silicon containing partners may not be included, namely pentavalent, anionic silicon groups. Further, reactions including homocoupling between aryl silanes and those derived from competition experiments with multiple nucleophilic functionalities may be also excluded. Similes string of reactants and products may be tabulated with other reaction components tabulated as numerical quantities or standardized component labels.

### Descriptor Calculation

In an exemplary embodiment, descriptors for reactants may be calculated using 128-bit Morgan Fingerprints calculated in RDkit. Product descriptors may not improve the models and may be therefore excluded. The exact implementation of this may be made publicly available in any of prediction packages. The reaction components may be then represented as a bit vector representing the presence of absence of that component. These may be calculated using in-house python scripts. The exact bit vector for each reaction component and their corresponding labels may be available in a conditions.csv file. For each reaction, the dimension of the reaction vector is 358.

### Model Generation

In an exemplary embodiment, various example models used in this study may be constructed using Keras with a Tensorflow backend. The basic architecture of all networks may be an input layer of 358 neurons, two hidden layers, and an output layer of a single neuron. Hyperparameters optimized include activation functions, percent dropout in the hidden layers, and number of nodes in the hidden layers. The optimizer used may be the adam optimizer and loss function may be mean squared error. To avoid overfitting, the models may be trained for only 20 epochs. Permissible activation functions may include Relu, Selu, and Elu functions. The number of possible nodes for the hidden layers may be constrained to the range 2-357 and the possible dropout for each layer ranged from 0.0-0.3.

The total dataset may be divided into 1110 training reactions, 185 validation reactions, and 155 test reactions. The models may be then optimized using a random search for hyperparameter selection with 1,000 iterations. The models may be then compared on the basis of their mean absolute error (MAE) in predicting the reaction outcomes for the validation set reactions. Using this metric, the top 100 neural networks may be selected to be used in the ensemble of neural networks used in the remainder of the workflow. A total summary of all hyperparameter combinations may be stored in a summary_file.csv file, and the summary of the models used in the ensemble may be stored in a best_models.csv. Further, .json files may be used for all models and 0.5 h files may contain the respective weights. The exemplary embodiment may be readily reproduced or adapted to new applications. Finally, a nnets folder may contain a summary performance of all models, not just those used in the final ensemble of models.

With the ensemble selected, the training, validation, and test sets may be calculated averaging the predictions for each reaction across the ensemble of 100 neural networks. Further, this same protocol may be used in the prediction of “future” reactions. Predicted vs observed values for each set may be stored in the final_pred_summary.xlsx file.

### Development of Recommendation Software

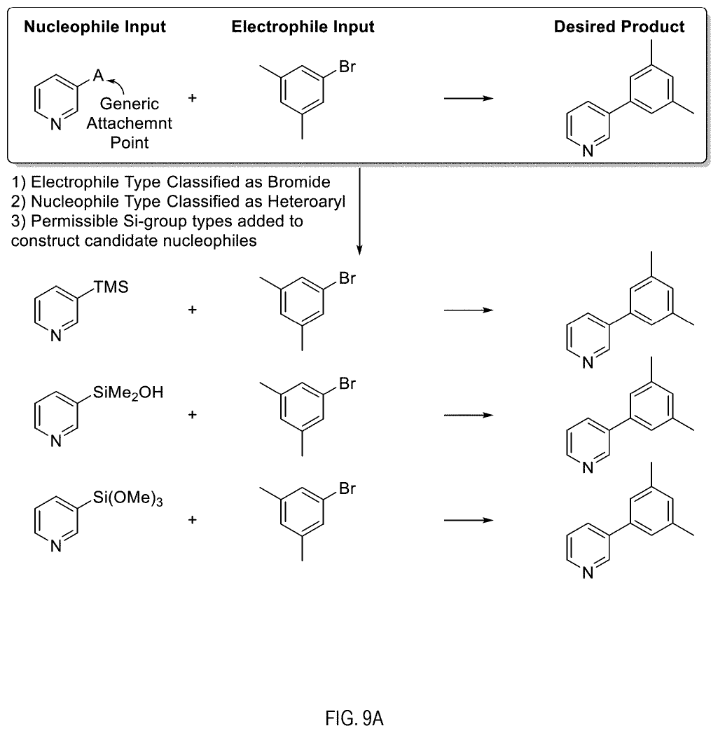

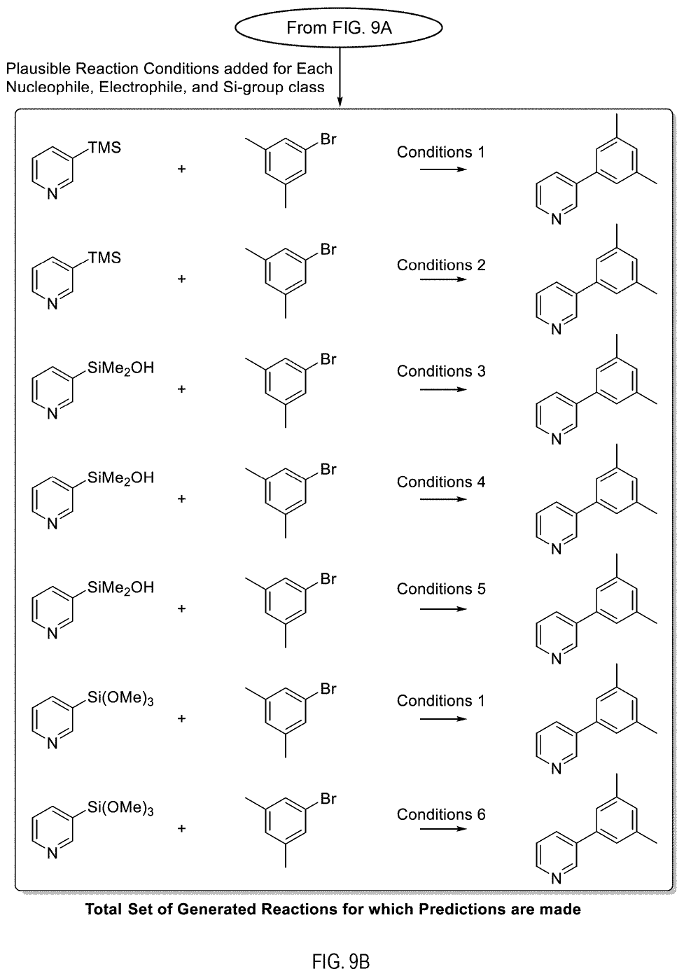

To constrain predictions to the domain of the model, software for systematically classifying reactions into categories may be developed. Once categorized, conditions proven to work for that specific category may be used in predictions, and the software aims to give a rank-order of these predictions may be given substrate combinations. First, reactions may be sorted by the identity of the electrophile as and iodide, bromide, chloride, or sulfonate. Note that the listed order is the order in which the software seeks to classify—a reactant containing both an iodide and a bromide may be classified as an iodide. Next, the nucleophile may be sorted as either an aryl, heteroaryl, alkenyl, or alkynyl transmetalating group. It is noteworthy that the software may be designed to take a generic attachment in place of a defined silicon group. The software then creates unique silicon nucleophiles that contain the desired transmetalating partner with different type of silicon-containing moieties (TMS-, HOMe₂Si—, OMe₃Si—, etc.). Silicon-containing groups that have been used successfully with that type of nucleophile may be included. Then, reaction conditions that are compatible with that specific nucleophile (e.g. the category and specific silicon group) and the electrophile may be constructed, turned into reaction vectors, and the yield is predicted. A general summary of this process for generating reactions to be predicted is illustrated in FIGS. 9A and 9B. In various embodiments, a silicon-containing group may also be referred as a silicon-containing moiety.

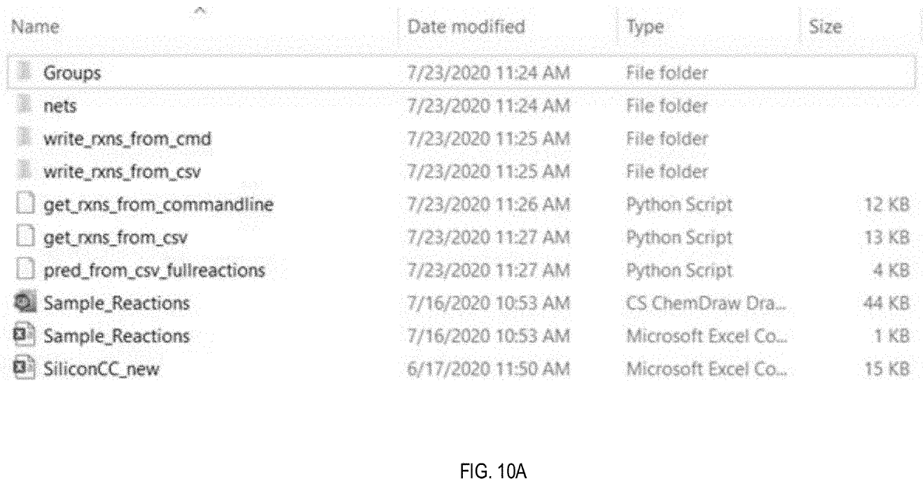

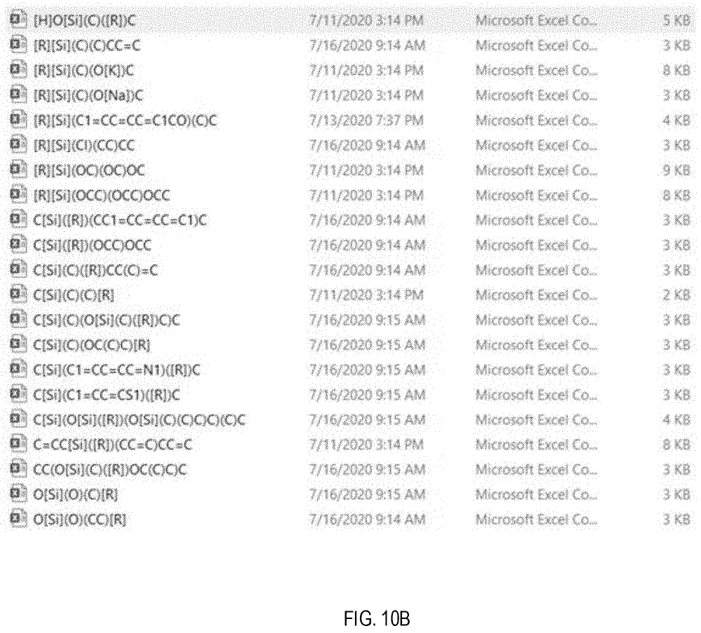

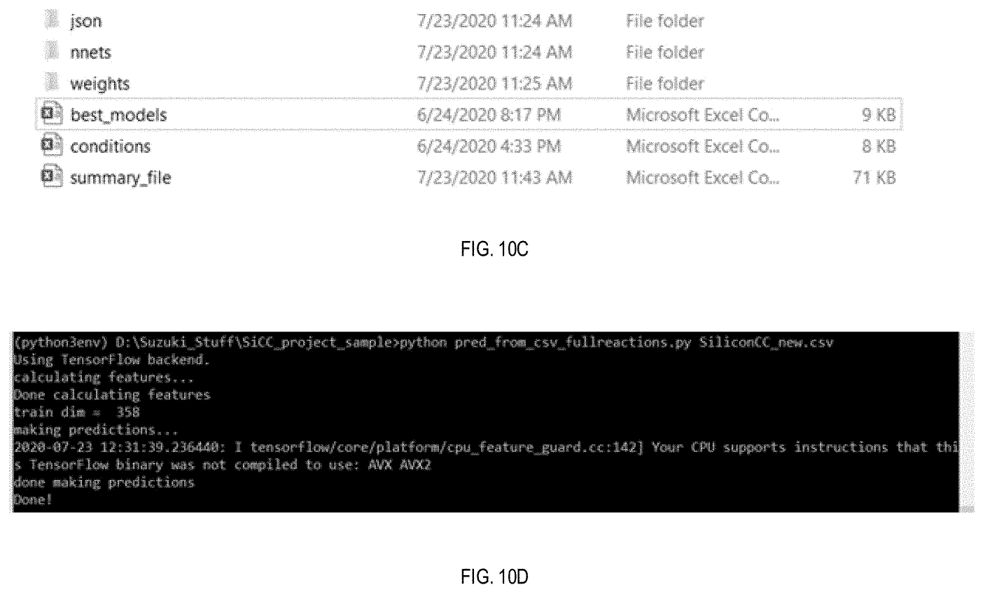

In an exemplary implementation, a sample folder with a pre-prepared file tree and multiple python scripts may be provided for different use cases. FIG. 10A shows an overview of the contents of this exemplary directory. The sample project folder may use a SiCC software.

The sample folder may be set up such that it can be copied and pasted and used immediately. The groups folder contains the different I, Br, CI, and SO2 subdirectories. These subdirectories contain the reaction conditions from the Organic Reactions (OR) chapter that have been used with the respective types of electrophiles. In each of these folders, there are alkenyl, alkynl, aryl, and heteroaryl directories. Each of these folders contains the identities of the silicon groups that can used to construct nucleophilic partners. The type of groups may be indicated by the names of the .csv file and are the smiles attached to a generic (R) substituent, as shown in FIG. 10B. The .csv files then contain examples of reactions from the OR chapter from which reaction conditions will be extracted and applied to the new electrophile/nucleophile pair.

Subdirectory containing different silicon group options may have names of csv files and plausible reaction conditions as the contents of those csv files.

The nets folder contains the neural networks to be loaded in the json subdirectory, the respective weights of those networks in the weights subdirectory. The nnets subdirectory may be a summary of each neural network generated during hyperparameter optimization. The file summary_file.csv contains hyperparameter and performance information for all networks, and the file best_networks.csv is what the code used to identify which networks to use to make future predictions. The file conditions.csv is what is used to provide reaction conditions descriptors for the construction of reaction vectors for new predictions, as shown in FIG. 10C.

The subdirectory may contain the models, the summary of hyperparameter optimization, the summary of the best models, and the file may contain bit vectors for reaction conditions.

The two other folders in the sample project directory may be used to store the generated in silico reactions prior to predicting their reaction outcomes. These are empty but will be populated after running one of two different submission scripts. In order to submit reaction for predictions, there are three different options. The first option is to provide the exact reactions one desires to test. Note that if a reaction component is included that is not in conditions.csv the code will crash as that reaction is not supported. It may, however, allow for prediction of novel coupling partners. As an example, the data for the prediction of future reactions may be provided in the SiliconCC_new.csv file. This provides the sample format for all reactions. In this case the experimental yields are known and have been included in the file, but for novel reactions 0 may be used as a placeholder in this column and only consider the predicted yields in the output file. To run these predictions, simply use the pred_from_csv_fullreactions.py file in the following manner, as shown in FIG. 10D.

A user may type “python pred_from_csv_fullreaction.py [name of file with reactions].csv” into command line, and without any modification, the code may make predictions for the contents of the .csv file. The csv file of interest and the python script may be in the same directory when it is run (setting up the directory exactly as in the example folder is recommended). When this is run, a new file appears which is called test_preds_fromcsv_futurepreds.csv. This file contains the predictions from the csv file. Upon examination of the file, columns for predicted yield and standard deviation in predicted yields may be added. For benchmarking purposes, the SiliconCC_new.csv file may be provided, and performing this sequence may give the same results as obtained for the “future” predictions as discussed above.

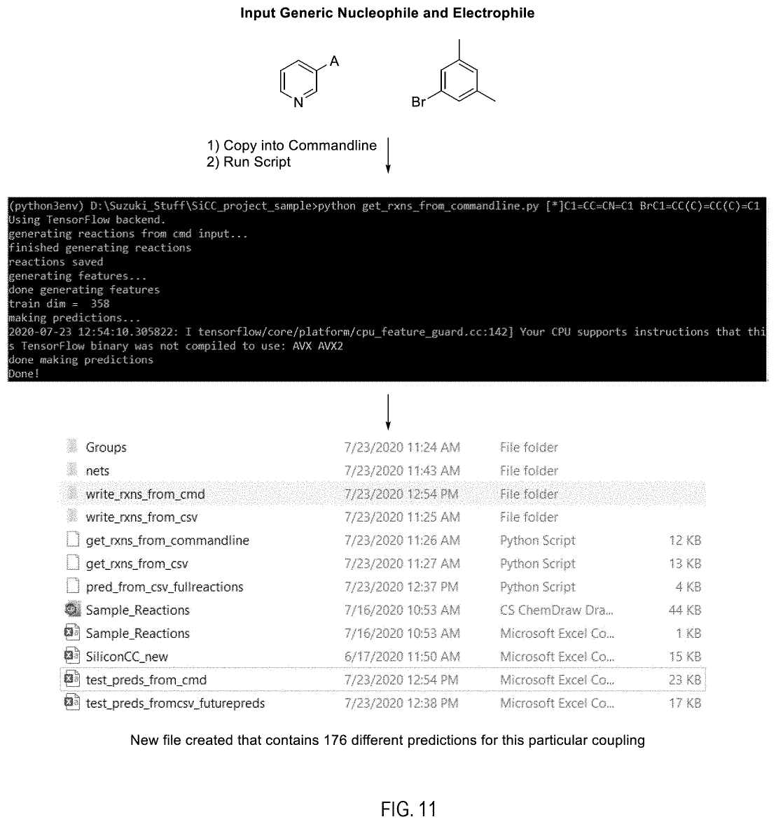

In another exemplary embodiment, another example system may predict the reaction outcome for the same transmetalating group but with many different silicon groups. This functionality may be accessed using the get_rxns_from_commandline.py and get_rxns_from_csv.py scripts. Both scripts work in a similar way as previously. For the first case, submission from commandline may be used to make predictions for a single desired coupling. In order to do this, a generic form of the nucleophile and the desired electrophile may be drawn in ChemDraw. Then, these groups may be copied as a SMILES string and pasted into commandline. Running this script may generate a new output file containing predictions for many different silicon groups with different reaction conditions. An overview of this process is depicted in FIG. 11.

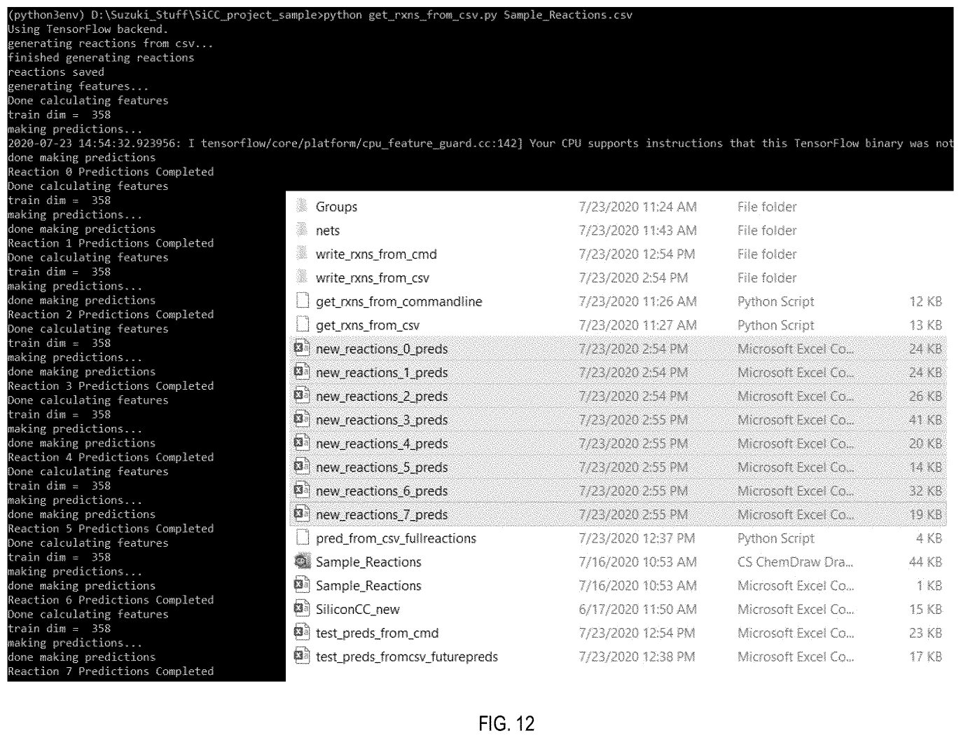

Alternatively or additionally, in the systems and methods described herein may be used for examining many potential reactions with different transmetalating partners or electrophiles. To enable this type of prediction, batch submission may be placed into a .csv file and run with the get_rxns_from_csv.py script. This submission is similar to the command line submission, but the transmetalating group and electrophile smiles strings are copies into an excel file, which is then given to the python script as an argument. An example of one such file is the Sample_Reactions.csv file in the sample project folder, and the identity of those reactions are given in the associated chemdraw. In this case, the submission syntax may be as depicted in FIG. 12. The program will construct reaction for each individual reactant combination present in the csv file and will give a unique output file for each reaction.

In various implementations, the machine learning models may be implemented within/into control systems for chemical systems. The models may be used to select conditions for real-world reactions as an output of the system. Thus, the models may be implemented in a practical system with the practical application of controlling chemical reactions to increase reaction success likelihood and/or reaction yield.

In some implementations, a multiple-tier reaction condition stack (RCS) may be implemented on reaction condition circuitry. In various implementations, the RCS may include an input tier, which may handle data input (e.g., from operators); a domain tier, which may handle classification of coupling partners into type-classes and/or subclasses; an condition tier, which may handle selection of candidate reaction conditions; a neural network tier which may handle interaction with neural networks; and/or a display tier which may handle data display on interface elements.

A stack may refer to a multi-tier computer architecture that defines the interaction of software and hardware resources at the multiple tiers. The Open Systems Interconnection (OSI) model is an example of a stack-type architecture. The tiers of a stack may pass data and hardware resources among themselves to facilitate data processing. As one example, for the RCS, the input tier may provide the display tier with hardware-based human interface device resources to for interactions with interface elements coordinated at the display tier. Hence, the input layer may provide a hardware resource, e.g., hardware-based human interface device resources, to the display tier. Accordingly, the multiple-layer stack architecture of the stack may improve the functioning of the underlying hardware. In various contexts, the tiers of such a stack may be interchangeably referred to as “layers.” However, for clarity and to avoid confusion with neural network layers, the term “tier” is used.

The present disclosure also includes various embodiments including a portion, all, or a combination of more than one below embodiments/implementations.

A1. A method including:

obtaining test input data for a test coupling partner of a test chemical type;

obtaining selected input data for a selected coupling partner of a selected chemical type, the selected chemical type being a counterpart to the test chemical type;

based on one or more entries in a reaction condition library:

optionally, determining a type-class for the test coupling partner, the selected coupling partner, or both;

optionally, determining a validity domain based on the type-class;

determining a candidate reaction condition set, the candidate reaction condition set optionally including a previous reaction condition used in a previous reaction involving a member of the test chemical type and a member of the selected chemical type; and

optionally, rejecting a second reaction condition set responsive to the second reaction condition set being outside the validity domain;

determining a candidate condition vector representative of the candidate reaction condition set;

comparing the candidate reaction condition set to previous reaction condition sets by applying the candidate condition vector to an input layer of a neural network; and

responsive to applying the candidate condition vector, receiving an output at an output layer of the neural network, the output indicative of an predicted yield from reacting the test coupling partner and the selected coupling partner under the candidate reaction condition set, where:

optionally, the method includes ranking the candidate reaction condition set based on the predicted yield relative to other possible reaction condition sets for the test coupling partner and selected coupling partner, where:

optionally, the other possible reaction condition sets are reaction conditions sets within the validity domain.

A2. The method of A1, where:

the test chemical type includes a nucleophile chemical type;

the selected chemical includes an electrophile chemical type.

A3. The method of any of the preceding methods, further including:

optionally, determining a test type-class for the test coupling partner, the test type-class including a type-class within the test chemical type; and

optionally, determining a selected type-class for the selected coupling partner, the selected type-class including a type-class within the selected chemical type, where:

optionally, determining the selected type-class, the test type-class, or both includes referencing the reaction condition library; and

optionally, determining the selected type-class, the test type-class, or both includes analyzing a chemical structure specified in the test input data, the selected input data, or both.

A4. The method of any of the preceding methods, where the test input data includes an unspecified silicon-containing moiety for the test coupling partner, where:

optionally, the method further includes determining one or more silicon-containing moieties compatible with a specified portion of the test coupling partner indicated by the test data, where:

optionally, determining the test type-class includes assigning a silicon-containing moiety to the test coupling partner.

A5. The method of A3 or any of the other preceding methods, where the method further includes assigning any coupling partner to a single type-class when that coupling partner is a member of two or more type-classes, where:

optionally, assigning any coupling partner to a single type-class includes assigning that coupling partner to a type-class including a highest ranked type-class of which that coupling partner is a member.

A6. The method of A5 or any of the other preceding methods, where the selected type-class includes bromine containing electrophiles, iodine containing electrophiles, chlorine containing electrophiles, sulfonate containing electrophiles, or any combination thereof, where:

optionally, iodine containing electrophiles are ranked above bromine containing electrophiles;

optionally, bromine containing electrophiles are ranked above chlorine containing electrophiles; and

optionally, chlorine containing electrophiles are ranked above sulfonate containing electrophiles.

A7. The method of A3, A4, A5 or any of the other preceding methods, where determining the test type-class includes determining whether the test coupling partner includes an aryl nucleophile, a heteroaryl nucleophile, an alkenyl nucleophile, or alkynyl nucleophile.

A8. The method of any of the preceding methods, where:

the method further includes:

subdividing the test type-class into test type-subclasses, where optionally:

determining test type-subclass for the test coupling partner includes determining a silicon-containing moiety for the test coupling partner; and

assigning the test coupling partner to a test type-class based on the silicon-containing moiety, where:

optionally, the silicon-containing moiety includes a trimethylsilyl silicon-containing moiety, a dimethyl silanol silicon-containing moiety, or a triallylsilyl silicon-containing moiety, or other silicon-containing moiety.

A9. The method of any of the preceding methods, where the previous reaction involved a member of the test type-class and a member of the selected type-class, where:

optionally, the previous reaction involved a member of the test type-subclass.

A10. The method of A9 or any of the preceding methods, further including selecting the candidate reaction set when the previous reaction involved a member of the test type-class and a member of the selected type-class and the reaction condition library includes no reactions with a member of the test type-subclass and a member of the selected type-class.

A11. The method of any of the preceding methods, where reaction condition sets for previous chemical reactions involving a member of the test type-class and a member of the selected type-class establish a validity domain for the test coupling partner and the selected coupling partner, where:

optionally, a non-domain reaction condition set for a non-domain reaction not involving a member of the test type-class and a member of the selected type-class is rejected based on the non-domain reaction condition set being outside the validity domain.

A12. The method of any of the preceding methods, where the test input data, the selected input data, or both include a simplified molecular-input line-entry system (SMILES) string or other line-entry string for a chemical model.

A13. The method of any of the preceding methods where the test input data, the selected input data, or both a multi-dimensional model of a chemical structure, where:

optionally, the method includes converting the multi-dimensional model to a line-entry string for a chemical model.

A14. The method of any of the preceding methods, where the previous reaction condition includes an equivalency, a Pd-source, a ligand, a base, a solvent, an additive, a reaction temperature, a product, and/or a yield.

A15. The method of any of the preceding methods, where the test coupling partner and the selected coupling partner couple via a sp2-sp2 coupling and/or a sp-sp2 coupling.

A16. The method of any of the preceding methods where:

optionally, the candidate reaction condition set includes multiple reaction conditions; and

optionally, the candidate reaction condition set indicates the presence or absence of one or more reaction conditions

optionally, the candidate reaction condition set includes one or more reaction conditions that specify the presence or absence of one or more reaction components.

A17. The method of any of the preceding methods where determining the candidate reaction vector includes assigning the candidate reaction condition set to one or more bit vectors indicating the presence or absence of reaction conditions, where:

optionally, where determining the candidate reaction vector includes concatenating the one or more bit vectors or adding the bit vectors in to an information-preserving data-structure;

optionally, assigning the bit vectors includes using virtually any encoding scheme that can represent the possible reaction conditions for the bit vectors;

optionally, assigning the bit vectors includes calculating 128-bit Morgan fingerprints for reactants;

optionally, assigning the bit vectors includes omitting descriptor for products;

optionally, assigning the bit vectors includes assigning a bit vector with a specific dimension, where:

optionally, the specific dimension is matched to the number of neurons in the input layer, for example 358 dimensions; and

optionally, assigning the bit vectors include using a one-hot/cold encoding scheme including encoding using a single first-state bit with other encoding bits in the other state, where:

optionally, the use of one-hot/cold encoding improves the performance of the underlying hardware by increasing processing speed by allowing for fewer processing cycles to be used in the detection of bit-vector coding errors, allowing for fewer processing cycles to be used in the detection of disallowed states, and/or facilitating increased central processing unit (CPU) clock speeds during analysis.

A18. The method of any of the preceding methods where the candidate reaction condition set includes an indication of the test coupling partner and/or the selected coupling partner.

A19. The method of any of the preceding methods, where the neural network is trained using reaction condition sets for previous chemical reactions in the reaction condition library.

A20. The method of any of the preceding methods, where the neural network includes one or more hidden layers between the input layer and output layer, where:

optionally, the neural network includes two hidden layers between the input and output layers.

A21. A method including:

structuring a neural network to include an input layer, an output layer, and one or more hidden layers between the input and output layers;

training, over multiple training epochs, the neural network to reproduce a reaction condition library describing reactants, reaction conditions, and yields, the reactants including test type-class members and selected type-class members, where:

optionally, the method further includes using the neural network in accord with any of the methods of any of the preceding claims.

A22. The method of A21, where training includes constructing the neural network on a platform including an application programming interface (API) coupled to a machine-learning backend, where:

optionally, the API includes Keras and/or the machine-learning backend includes TensorFlow.

A23. The method of A21 or A22, where training includes optimizing the number, structure (e.g., interconnects), and/or behavior (e.g. percent dropout, activation functions) of nodes in the one or more hidden layers, where:

optionally, permissible activation functions include relu, selu, and elu functions.

A24. The method of any of the preceding methods, where the method further includes using any interface element described in the specification to implement the method.

A25. The method of any of the preceding methods, where the method is performed using a multiple-tier reaction condition stack (RCS), where:

optionally, the test input data and selected input data are obtained at an input tier of the RCS;

optionally, the test type-class, selected type-class, and/or test type-subclass are determined at a domain tier of the RCS;

optionally, the candidate reaction condition set is determined at a condition tier of the RCS;

optionally, interactions with the neural network at performed at a neural network tier of the RCS;

optionally, displays of inputs, outputs, or other data are coordinated and displayed using interface elements generated at a display tier of the RCS; and

optionally, the RCS improves the operation of the underlying hardware by the multiple tiers passing data and hardware resources among themselves to facilitate data processing.

A26. A system including reaction condition circuitry configured to implement any of the methods of any of the preceding claims.

A27. A product including:

machine-readable media; and

instructions stored on the machine-readable media, the instructions configured to cause a machine to execute the method of any one of claims 1 to 25, where:

optionally, the machine-readable media is non-transitory;

optionally, the machine-readable media is other than a transitory signal; and

optionally, the instructions are executable.

A28. A method including implementing any of or any combination of the features described in the above disclosure and methods/products/systems.

A29. A system including circuitry configured to implement any of or any combination of the features described in the above disclosure and methods/products/systems.

A30. A product including:

machine-readable media; and

instructions stored on the machine-readable media, the instructions configured to cause a machine to implement any of or any combination of the features described in the above disclosure and claims, where:

optionally, the machine-readable media is non-transitory;

optionally, the machine-readable media is other than a transitory signal; and

optionally, the instructions are executable.

While the particular disclosure has been described with reference to illustrative embodiments, this description is not meant to be limiting. Various modifications of the illustrative embodiments and additional embodiments of the disclosure will be apparent to one of ordinary skill in the art from this description. Those skilled in the art will readily recognize that these and various other modifications can be made to the exemplary embodiments, illustrated and described herein, without departing from the spirit and scope of the present disclosure. It is therefore contemplated that the appended claims will cover any such modifications and alternate embodiments. Certain proportions within the illustrations may be exaggerated, while other proportions may be minimized. Accordingly, the disclosure and the figures are to be regarded as illustrative rather than restrictive.

What is claimed is:

1. A method for using a neural network set to determine a reaction condition, the method performed by a device comprising a memory storing instructions and a processor in communication with the memory, the method comprising:
obtaining test input data for a test coupling partner of a test chemical type;
obtaining selected input data for a selected coupling partner of a selected chemical type, the selected chemical type being a counterpart to the test chemical type;
determining, based on a reaction condition library comprising at least one entry, a candidate reaction condition set according to the test input data and selected input data, the candidate reaction condition set comprising a previous reaction condition;
determining a candidate reaction vector representative of the candidate reaction condition set;
inputting the candidate reaction vector into an input layer of a neural network set; and
receiving an output at an output layer of the neural network set, the output indicative of a predicted yield from reacting the test coupling partner and the selected coupling partner under the candidate reaction condition set.

2. The method according to claim 1, wherein:
the test chemical type comprises a nucleophile chemical type;
the selected chemical comprises an electrophile chemical type.

3. The method according to claim 1, further comprising:
determining a test type-class for the test coupling partner, the test type-class comprising a type-class within the test chemical type; and
determining a selected type-class for the selected coupling partner, the selected type-class comprising a type-class within the selected chemical type, wherein:
determining the selected type-class, the test type-class, or both comprises referencing the reaction condition library; and/or
determining the selected type-class, the test type-class, or both comprises analyzing a chemical structure specified in the test input data, the selected input data, or both.

4. The method according to claim 3, wherein:
in response to the test coupling partner is a member of two or more type-classes, the test type-class is a highest ranked type-class of which that the test coupling partner is a member; and/or
in response to the selected coupling partner is a member of two or more type-classes, the selected type-class is a highest ranked type-class of which that the selected coupling partner is a member.

5. The method according to claim 4, wherein:
the selected type-class comprises at least one of the following: bromine containing electrophiles, iodine containing electrophiles, chlorine containing electrophiles, sulfonate containing electrophiles, or any combination thereof, wherein:
the iodine containing electrophiles are ranked above the bromine containing electrophiles;
the bromine containing electrophiles are ranked above the chlorine containing electrophiles; and
the chlorine containing electrophiles are ranked above the sulfonate containing electrophiles.

6. The method according to claim 3, wherein:
the test type-class comprises at least one of the following: an aryl nucleophile, a heteroaryl nucleophile, an alkenyl nucleophile, or alkynyl nucleophile.

7. The method according to claim 3, wherein:
the previous reaction involves a member of the test type-class and a member of the selected type-class.

8. The method according to claim 1, wherein:
the test input data, the selected input data, or both comprise a simplified molecular-input line-entry system (SMILES) string or other line-entry string for a chemical model.

9. The method according to claim 1, wherein the previous reaction condition comprises an equivalency, a Pd-source, a ligand, a base, a solvent, an additive, a reaction temperature, a product, and/or a yield.

10. The method according to claim 1, wherein the determining the candidate reaction vector comprises:
assigning the candidate reaction condition set to bit vectors indicating a presence or absence of reaction conditions by calculating 128-bit Morgan fingerprints for reactants,
concatenating the bit vectors to obtain an information-preserving data-structure to obtain the candidate reaction vector.

11. The method according to claim 1, wherein:
the neural network set comprises at least one neural network, and
each of the at least one neural network comprises two hidden layers between the input layer and the output layer, and the input layer comprises 358 neurons.

12. An apparatus for using a neural network set to determine a reaction condition, the apparatus comprising:
a memory storing instructions; and
a processor in communication with the memory, wherein, when the processor executes the instructions, the processor is configured to cause the apparatus to perform:
obtaining test input data for a test coupling partner of a test chemical type,
obtaining selected input data for a selected coupling partner of a selected chemical type, the selected chemical type being a counterpart to the test chemical type,
determining, based on a reaction condition library comprising at least one entry, a candidate reaction condition set according to the test input data and selected input data, the candidate reaction condition set comprising a previous reaction condition,
determining a candidate reaction vector representative of the candidate reaction condition set,
inputting the candidate reaction vector into an input layer of a neural network set, and
receiving an output at an output layer of the neural network set, the output indicative of a predicted yield from reacting the test coupling partner and the selected coupling partner under the candidate reaction condition set.

13. The apparatus according to claim 12, wherein:
the test chemical type comprises a nucleophile chemical type;
the selected chemical comprises an electrophile chemical type.

14. The apparatus according to claim 12, further comprising:
determining a test type-class for the test coupling partner, the test type-class comprising a type-class within the test chemical type; and
determining a selected type-class for the selected coupling partner, the selected type-class comprising a type-class within the selected chemical type, wherein:
determining the selected type-class, the test type-class, or both comprises referencing the reaction condition library; and/or
determining the selected type-class, the test type-class, or both comprises analyzing a chemical structure specified in the test input data, the selected input data, or both.

15. The apparatus according to claim 12, wherein:
the test input data, the selected input data, or both comprise a simplified molecular-input line-entry system (SMILES) string or other line-entry string for a chemical model.

16. The apparatus according to claim 12, wherein:
the neural network set comprises at least one neural network, and
each of the at least one neural network comprises two hidden layers between the input layer and the output layer, and the input layer comprises 358 neurons.

17. A non-transitory computer readable storage medium storing computer readable instructions, wherein, the computer readable instructions, when executed by a processor, are configured to cause the processor to perform:
obtaining test input data for a test coupling partner of a test chemical type;
obtaining selected input data for a selected coupling partner of a selected chemical type, the selected chemical type being a counterpart to the test chemical type;
determining, based on a reaction condition library comprising at least one entry, a candidate reaction condition set according to the test input data and selected input data, the candidate reaction condition set comprising a previous reaction condition;
determining a candidate reaction vector representative of the candidate reaction condition set;
inputting the candidate reaction vector into an input layer of a neural network set; and
receiving an output at an output layer of the neural network set, the output indicative of a predicted yield from reacting the test coupling partner and the selected coupling partner under the candidate reaction condition set.

18. The non-transitory computer readable storage medium according to claim 17, wherein:
the test chemical type comprises a nucleophile chemical type;
the selected chemical comprises an electrophile chemical type.

19. The non-transitory computer readable storage medium according to claim 17, further comprising:
determining a test type-class for the test coupling partner, the test type-class comprising a type-class within the test chemical type; and
determining a selected type-class for the selected coupling partner, the selected type-class comprising a type-class within the selected chemical type, wherein:
determining the selected type-class, the test type-class, or both comprises referencing the reaction condition library; and/or
determining the selected type-class, the test type-class, or both comprises analyzing a chemical structure specified in the test input data, the selected input data, or both.

20. The non-transitory computer readable storage medium according to claim 17, wherein:
the neural network set comprises at least one neural network, and
each of the at least one neural network comprises two hidden layers between the input layer and the output layer, and the input layer comprises 358 neurons.
